SEQUENCE LISTING (91 sequences)

SEQ ID NO: 1 - protein, 8 aa, synthetic construct
GHTSRTYA

SEQ ID NO: 2 - protein, 8 aa, synthetic construct
GRTFRTYA

SEQ ID NO: 3 - protein, 8 aa, synthetic construct
GRTFRTYS

SEQ ID NO: 4 - protein, 8 aa, synthetic construct
GRTFRIYA

SEQ ID NO: 5 - protein, 8 aa, synthetic construct
TSFFSPSA

SEQ ID NO: 6 - protein, 8 aa, synthetic construct
ASFFSPSA

SEQ ID NO: 7 - protein, 8 aa, synthetic construct
GFAFSSYY

SEQ ID NO: 8 - protein, 8 aa, synthetic construct
GFTFSSYW

SEQ ID NO: 9 - protein, 8 aa, synthetic construct
GFTFSRYG

SEQ ID NO: 10 - protein, 8 aa, synthetic construct
GFPFKHYW

SEQ ID NO: 11 - protein, 8 aa, synthetic construct
GRTFSTYN

SEQ ID NO: 12 - protein, 8 aa, synthetic construct
GRSFSTKT

SEQ ID NO: 13 - protein, 8 aa, synthetic construct
GSVVSSNA

SEQ ID NO: 14 - protein, 5 aa, synthetic construct
GRLYS

SEQ ID NO: 15 - protein, 8 aa, synthetic construct
NFLSSRFE

SEQ ID NO: 16 - protein, 8 aa, synthetic construct
GFTFSSAT

SEQ ID NO: 17 - protein, 8 aa, synthetic construct
GSIFKVAG

SEQ ID NO: 18 - protein, 8 aa, synthetic construct
GFTFSNYA

SEQ ID NO: 19 - protein, 8 aa, synthetic construct
GNIFSPNA

SEQ ID NO: 20 - protein, 7 aa, synthetic construct
IGWNGGT

SEQ ID NO: 21 - protein, 7 aa, synthetic construct
IGWNSGT

SEQ ID NO: 22 - protein, 7 aa, synthetic construct
IGWNAGT

SEQ ID NO: 23 - protein, 7 aa, synthetic construct
ITRGGTT

SEQ ID NO: 24 - protein, 8 aa, synthetic construct
INSGGGTT

SEQ ID NO: 25 - protein, 8 aa, synthetic construct
INRGGIIT

SEQ ID NO: 26 - protein, 8 aa, synthetic construct
ISWDGGTT

SEQ ID NO: 27 - protein, 8 aa, synthetic construct
VNNDGGGR

SEQ ID NO: 28 - protein, 8 aa, synthetic construct
ISWSGNST

SEQ ID NO: 29 - protein, 7 aa, synthetic construct
ISSSGIT

SEQ ID NO: 30 - protein, 7 aa, synthetic construct
ITRSGLE

SEQ ID NO: 31 - protein, 7 aa, synthetic construct
IFRDGNT

SEQ ID NO: 32 - protein, 8 aa, synthetic construct
ITDDGRDT

SEQ ID NO: 33 - protein, 7 aa, synthetic construct
ITSGGGT

SEQ ID NO: 34 - protein, 8 aa, synthetic construct
ITNDGLRT

SEQ ID NO: 35 - protein, 7 aa, synthetic construct
ITSSGLT

SEQ ID NO: 36 - protein, 13 aa, synthetic construct
AADDAKVLIVPHY

SEQ ID NO: 37 - protein, 13 aa, synthetic construct
AADDSKVLIVPHY

SEQ ID NO: 38 - protein, 13 aa, synthetic construct
AADDAKVLIVPHF

SEQ ID NO: 39 - protein, 13 aa, synthetic construct
AADDSKVLIVPVY

SEQ ID NO: 40 - protein, 13 aa, synthetic construct
AADDSQVLIVPHY

SEQ ID NO: 41 - protein, 13 aa, synthetic construct
AADDAKVLIVPNY

SEQ ID NO: 42 - protein, 9 aa, synthetic construct
NLKSIDLQW

SEQ ID NO: 43 - protein, 9 aa, synthetic construct
NLKSINLQW

SEQ ID NO: 44 - protein, 10 aa, synthetic construct
VAGEKGTKDY

SEQ ID NO: 45 - protein, 19 aa, synthetic construct
ATAPTTWGKQPYQGPGYKT

SEQ ID NO: 46 - protein, 14 aa, synthetic construct
AATPRPRLGQGSDY

SEQ ID NO: 47 - protein, 14 aa, synthetic construct
AKGWLVSDYDQVTY

SEQ ID NO: 48 - protein, 15 aa, synthetic construct
AAHSSSRATITSPDY

SEQ ID NO: 49 - protein, 8 aa, synthetic construct
EKVDGMYA

SEQ ID NO: 50 - protein, 9 aa, synthetic construct
RTRGPNGDY

SEQ ID NO: 51 - protein, 9 aa, synthetic construct
HVHILGRDY

SEQ ID NO: 52 - protein, 12 aa, synthetic construct
YADVTAGGPQRY

SEQ ID NO: 53 - protein, 14 aa, synthetic construct
SAQGADSNYALFRS

SEQ ID NO: 54 - protein, 14 aa, synthetic construct
GVRGTSRWGRSFRS

SEQ ID NO: 55 - protein, 15 aa, synthetic construct
KWAVFRDYGMGADDY

SEQ ID NO: 56 - protein, 119 aa, synthetic construct
  VARIANT 109: note = Glutamine or Tryptophan
QVQLQEAGGGLVQAGGSLRLSCAASGHTSRTYAMGWFRQAPGKEREFVARIGWNGGTYYADFVKGRFTISRDGAKNTVYLQMNSLKPEDTAVYYCAADDAKVLIVPHYXGQGTQVTVSS

SEQ ID NO: 57 - protein, 119 aa, synthetic construct
QVQLQESGGGLVQAGGSLRLSCAASGRTFRTYAMGWFRQAPGKEREFVARIGWNSGTYYADSVKGRFTISRDGAKNTVYLQMNSLKPEDTAVYYCAADDSKVLIVPHYWGQGTQVTVSS

SEQ ID NO: 58 - protein, 119 aa, synthetic construct
QVQLQESGGGLVQAGGSLRLSCAASGRTFRTYAMGWFRQAPGKEREFVARIGWNGGTYYADSVKGRFTISRDGAENTVYLQMNSLKPEDTAVYYCAADDAKVLIVPHFWGQGTQVTVSS

SEQ ID NO: 59 - protein, 119 aa, synthetic construct
QVQLQESGGGLVQAGGSLRLSCAASGRTFRTYAMGWFRQAPGKEREFVARIGWNGGTYYVDSVKGRFTISRDGAKNTVYLQMNSLKPEDTAVYYCAADDSKVLIVPHYWGQGTQVTVSS

SEQ ID NO: 60 - protein, 119 aa, synthetic construct
QVQLQESGGGLVQPGDSLSLSCAASGRTFRTYSMGWFRQAPGKEREFVARIGWNGGTYYADSVKGRFTISRDGAKNTVYLQMNSLRPEDTAIYYCAADDSKVLIVPVYWGQGTQVTVSS

SEQ ID NO: 61 - protein, 119 aa, synthetic construct
QVQLQESGGGLVQAGGSLRLSCAASGRTFRTYAMGWFRQAPGKEREFVARIGWNSGTHYADSVKGRFTISRDGAKNTVYLQMNSLTPEDTAVYYCAADDSQVLIVPHYWGQGTQVTVSS

SEQ ID NO: 62 - protein, 119 aa, synthetic construct
QVQLQESGGGLVQAGGSLRLSCAASGRTFRTYSMGWFRQAPGKEREFVARIGWNGGTYYADSVKGRFTISRDGAKNTVYLQMNSLRPEDTAIYYCAADDSKVLIVPVYWGQGTQVTVSS

SEQ ID NO: 63 - protein, 119 aa, synthetic construct
QVQLQESGGGLVQAGGSLRLSCTASGRTFRTYAMGWFRQAPGKEREFVARIGWNGGTYYTDSVKGRFTISRDGAKNTVWLQMDSLKPEDTAVYYCAADDAKVLIVPNYWGQGTQVTVSS

SEQ ID NO: 64 - protein, 119 aa, synthetic construct
  VARIANT 36: note = Glutamine or Tryptophan
QVQLQESGGGLVQAGDSLRLSCAVSGHTSRTYAMGXFRQAPGKEREFVARIGWNGGTYYADFVKGRFTISRDGAKNLVWLQMNSLKPEDTAVYYCAADDAKVLIVPNYWGQGTQVTVSS

SEQ ID NO: 65 - protein, 119 aa, synthetic construct
QVQLVESGGGLVQAGGSLRLSCAASGRTFRTYSMGWFRQAPGKEREFVARIGWNGGTYYADSVKGRFTISRDGAKNTVYLQMNSLRPEDTAIYYCAADDSKVLIVPVYWGQGTQVTVSS

SEQ ID NO: 66 - protein, 119 aa, synthetic construct
QVQLQESGGGLVQAGGSLRLSCAASGRTFRTYAMGWFRQAPGKEREFVARIGWNGGTYYADSVKGRFTISRDGAKNTVYLQMNSLKPEDTAVYYCAADDSKVLIVPHYWGQGTQVTVSS

SEQ ID NO: 67 - protein, 119 aa, synthetic construct
QVQLQESGGGLVQAGGSLRLSCAASGRTFRIYAMGWFRQAPGKEREFVARIGWNAGTYVADSVKGRFTISRDGTKNTVYLQMNSLKPEDTAVYYCAADDSQVLIVPHYWGQGTQVTVSS

SEQ ID NO: 68 - protein, 115 aa, synthetic construct
QVQLQESGGGLVQAGGSLRLSCAASTSFFSPSAMGWYRQAPGKQRELVATITRGGTTNYADSVKGRFTISKDNADNTVFLQMNSLKPDDTAVYYCNLKSIDLQWWGQGTQVTVSS

SEQ ID NO: 69 - protein, 115 aa, synthetic construct
QVQLQESGGGLVQAGGSLRLSCAASASFFSPSAMGWYRQAPGKQRELVAIITRGGTTNYADSVKGRFTISKNNADNTVFLQMNSLKPDDTAVYYCNLKSINLQWWGQGTQVTVSS

SEQ ID NO: 70 - protein, 117 aa, synthetic construct
QVQLQESGGGNVQPGGSLRLSCAASGFAFSSYYMYWVRQAPGKGLEWVSIINSGGGTTTYADSVKGRFTISRDNAKNTLYLQMNSLKPEDTAVYYCVAGEKGTKDYWGQGTQVTVSS

SEQ ID NO: 71 - protein, 117 aa, synthetic construct
QVQLQESGGGVVQPGGSLRLSCAASGFTFSSYWMYWVRQTPGKGLEWVSLINRGGIITYYSDSVKGRFTISRDNAKNTVYLQMNSLKPEDTAVYYCVAGEKGTKDYWGQGTQVTVSS

SEQ ID NO: 72 - protein, 126 aa, synthetic construct
QVQLQESGGGLVQAGGSLRLSCAASGFTFSRYGMVWFRQSPGKEREIVAAISWDGGTTYYADSVKGRFTISRHNAKDTVDLQMNSLNPEDTAVYYCATAPTTWGKQPYQGPGYKTWGQGTQVTVSS

SEQ ID NO: 73 - protein, 126 aa, synthetic construct
QVQLQESGGGLVQAGGSLRLSCAASGFTFSRYGMVWFRQSPGKERELVSAISWDGGTTYYVDSVKGRFTISRDNAVDTVYLQMNSLKPEDTAVYYCATAPTTWGKQPYQGPGYKTWGQGTQVTVSS

SEQ ID NO: 74 - protein, 121 aa, synthetic construct
  VARIANT 13: note = Glutamine or Tryptophan
QVQLQESGGGLVXPGGSLRLSCVTSGFPFKHYWMYWVRQAPGKGVEWVSIVNNDGGGRHYSDSVKGRFTISRDNDKNVLYLEMNNLKPEDTARYYCAATPRPRLGQGSDYWGQGTQVTVSS

SEQ ID NO: 75 - protein, 121 aa, synthetic construct
QVQLQESGGGLVQAGGSLRLSCAASGRTFSTYNMAWFRQAPEKEREIVAVISWSGNSTYYADSVKGRFTISRDNAKNTVYLQMNSLKPEDTAVYYCAKGWLVSDYDQVTYWGQGTQVTVSS

SEQ ID NO: 76 - protein, 121 aa, synthetic construct
QVQLQESGGGLVQAGGSLRLSCAASGRSFSTKTMGWFRQAPGKERELVAAISSSGITYYADSMKGRFTISRDNARNMVFLQMNNLNPEDTAVYFCAAHSSSRATITSPDYWGQGTQVTVSS

SEQ ID NO: 77 - protein, 114 aa, synthetic construct
QVQLQESGGGSVQAGGSLRLSCVVSGSVVSSNAMGWYGQAPGKQRELVAWITRSGLENYKDSVRGRFTISRDNAKNTAYLQMDNLRPEDTAVYYCEKVDGMYARGQGTQVTVSS

SEQ ID NO: 78 - protein, 113 aa, synthetic construct
QVQLQESGGGLVQAGGSLRLSCAPSGRLYSVAWFRQRSGKEREFVSSIVGSYGSTFYADSVKGRFTISRDNAKNMLYLQMDSLKPEDTAVYYCRTRGPNGDYWGQGTQVTVSS

SEQ ID NO: 79 - protein, 115 aa, synthetic construct
  VARIANT 44: note = Glutamine or Tryptophan
QVQLQESGGGLVQAGESLRLSCAGTNFLSSRFEMGWYRQIPGEXRELVARIFRDGNTDYTDSVRGRFTISRDTAKNTIDLQMNNLKPEDSAGYLCHVHILGRDYWGQGTQVTVSS

SEQ ID NO: 80 - protein, 119 aa, synthetic construct
QVQLQESGGGLVQPGGSLRLSCATSGFTFSSATMIWVRQAPGKGPEWVAIITDDGRDTAYAASVQGRFTIARDNAKNMLYLQMNSLKSDDTAVYQCYADVTAGGPQRYWGQGTQVTVSS

SEQ ID NO: 81 - protein, 120 aa, synthetic construct
QVQLQESGGGLVQSGGSLRLSCAVSGSIFKVAGMQWYRQAPEKQRELVASITSGGGTNYADSVKGRFTISRDNAKNTAYLQMNSLTPEDTAVYFCSAQGADSNYALFRSRGQGTQVTVSS

SEQ ID NO: 82 - protein, 121 aa, synthetic construct
QVQLQESGGGLVQPGGSLRLSCTASGFTFSNYAMDWVRQAPGKGLEWVAAITNDGLRTEYEDTVQGRFTISRDNAKNTLYLQMNSLKPEDTAVYYCGVRGTSRWGRSFRSWGQGTQVTVSS

SEQ ID NO: 83 - protein, 121 aa, synthetic construct
QVQLQESGGGLVQPGGSLRLSCVASGNIFSPNAVGWYRQAPGKQRESVADITSSGLTRYADDVKGRFVISRDNAKGTVDLQMNSLKPEDTAVYYCKWAVFRDYGMGADDYWGQGTQVTVSS

SEQ ID NO: 84 - protein, 19 aa, synthetic construct
AAAYPYDVPDYGSHHHHHH

SEQ ID NO: 85 - protein, 6 aa, synthetic construct
STMVRS

SEQ ID NO: 86 - protein, 233 aa, Homo sapiens
DKTHTCPPCPAPELLGGPSVFLFPPKPKDTLMISRTPEVTCVVVDVSHEDPEVKFNWYVDGVEVHNAKTICPREEQYNSTYRVVSVLTVLHQDWLNGICEYKCKVSNICALPAPIEKTISICAKGOPREPOVYTLPPSREEMTICNOVSLTCLVKGFYPSDIAVEWESNGOPENNYKTITTVLDSDGSFFLYSICLTVDKSRWQQGNVFSCSVMHEALHNHYTQKSLSLSPGK

SEQ ID NO: 87 - protein, 6 aa, synthetic construct
HHHHHH

SEQ ID NO: 88 - protein, 15 aa, synthetic construct
EPKSCDKTHTCPPCP

SEQ ID NO: 89 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 13 aa, synthetic construct
VPRDCGCKPCICT

SEQ ID NO: 91 - protein, 214 aa, Mus musculus
VPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

(19) United States
(12) Patent Application Publication
(10) Pub. No.: US 2025/0289875 A1
(43) Pub. Date: Sep. 18, 2025

(54) GRP78 NANOBODIES

(71) Applicant: ACTINIUM PHARMACEUTICALS, INC., New York, NY (US)

(72) Inventors: GHOLAMREZA HASSANZADEH GHASSABEH, Sint-Genesius-Rode (BE); STEVE SCHOONOOGHE, Holsbeek (BE); HELEN KOTANIDES, NORWALK, CT (US)

(73) Assignee: ACTINIUM PHARMACEUTICALS, INC., New York, NY (US)

(21) Appl. No.: 18/862,655

(22) PCT Filed: May 10, 2023
(86) PCT No.: PCT/US2023/066837

§ 371 Date: Nov. 4, 2024

Related U.S. Application Data
(60) Provisional application No. 63/340,266, filed on May 10, 2022.

(51) Int. Cl. C07K 16/18; A61K 51/10

(52) U.S. Cl. CPC C07K 16/18; A61K 51/1018; A61K 51/1093; A61K 2121/00; C07K 2317/565; C07K 2317/569

(57) ABSTRACT

Provided are nanobodies that recognize human cell surface GRP78 protein, pharmaceutical compositions that include the nanobodies, and uses thereof.

### CROSS-REFERENCE TO RELATED APPLICATIONS

This application claims priority to U.S. provisional application Ser. No. 63/340,266 filed May 10, 2022, which is hereby incorporated by reference in its entirety.

### SEQUENCE LISTING

The instant application contains a Sequence Listing which has been submitted electronically in XML format and is hereby incorporated by reference in its entirety. Said XML copy, created on May 9, 2023 is named ATNM-021PCT_SL_ST26.xml and is 84,743 bytes in size.

### FIELD OF THE INVENTION

The presently claimed invention relates to the field of nanobodies.

### BACKGROUND

Glucose-regulated protein 78 (GRP78), also known as Endoplasmic reticulum chaperone BiP, is a heat shock protein 70 (HSP70) family molecular chaperone normally located in the lumen of the endoplasmic reticulum (ER) that binds newly synthesized proteins as they are translocated into the ER, maintaining them in a state of competence for subsequent folding and oligomerization. GRP78 is also a component of the translocation machinery, playing a role in retrograde transport across the ER membrane of aberrant proteins destined for degradation by the proteasome. GRP78 is an abundant protein under all growth conditions, but its synthesis is markedly increased under conditions that lead to the accumulation of unfolded polypeptides in the ER. Although generally intracellular, in tumor cells and cells undergoing stress, GRP78 is presented on the cell surface (cell surface GRP78, csGRP78). csGRP78 is significantly expressed on proliferating cancer cells, cancer stem cells, metastatic cancer cells, tumor-associated endothelium, cells in the tumor microenvironment, and cells undergoing various forms of stress such as severe glucose starvation (metabolic stress), lactic acidosis, hypoxia, and genotoxic stress such as from exposure to ionizing radiation or DNA damaging agents.

What is needed and provided by the various aspects of the present invention are new anti-huGRP78 nanobodies and targeting agents against cell surface GRP78.

### SUMMARY OF THE INVENTION

One aspect of the invention provides a protein that includes a human-GRP78 (huGRP78) binding, such as [human csGRP78]-binding, nanobody amino acid sequence which includes:
(i) the nanobody CDRs (CDR1, CDR2 and CDR3) of any of the nanobody clones set forth in FIG. 1A or FIG. 1B or otherwise disclosed herein;
(ii) one or more of the framework regions (FR1, FR2, FR3 and FR4) and the CDRs of any of the nanobody clones set forth in FIG. 1A or FIG. 1B or otherwise disclosed herein; and/or
(iii) the full nanobody sequence of any of the nanobody clones set forth in FIG. 1A or FIG. 1B, or otherwise disclosed herein.

The protein may, for example, be a monomeric nanobody (VHH), a dimeric or multimeric nanobody wherein the nanobody components are the same or at least some are different and which may have the same or different binding specificities, or a fusion protein including a nanobody sequence and another protein sequence, such as a nanobody-Fc fusion protein or a nanobody-epitope tag fusion protein.

The protein may, for example, be used for the treatment of a GRP78-expressing cancer in a mammalian subject such as a human patient. The protein may, for example, be radiolabeled and/or drug-conjugated for use in the treatment of a GRP78-expressing cancer in a mammalian subject such as a human patient. The protein may, for example, be radiolabeled for use in detecting and/or imaging GRP78-expressing cancer cells and/or tumors in a mammalian subject such as a human patient.

Additional features, advantages, and aspects of the invention may be set forth or apparent from consideration of the following detailed description, drawings if any, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1A sets forth the clone name, clone ID, CDR3 group, full VHH sequence and CDR sequences (delineated according to the IMGT system), ELISA huGRP78 binding data, and bio-layer interferometry (BLI) huGRP78 off-rate data for 18 anti-huGRP78 nanobody clones.

FIG. 1B set forth the clone name, clone ID, CDR3 group, full VHH sequence and CDR sequences (delineated according to the IMGT system), ELISA huGRP78 binding data, and bio-layer interferometry (BLI) huGRP78 off-rate data for 10 anti-huGRP78 nanobody clones.

FIG. 2 shows the qMFI flow cytometric cell-binding data for two human anti-huGRP78 nanobody clones showing specific binding to the HL60 human AML cell line.

### DETAILED DESCRIPTION

A nanobody (Nb) or VHH domain antibody is the variable region of a camelid heavy chain-only antibody. The present invention provides nanobodies and nanobody fusion proteins that specifically bind human GRP78 (huGRP78) and related compositions and methods of use thereof.

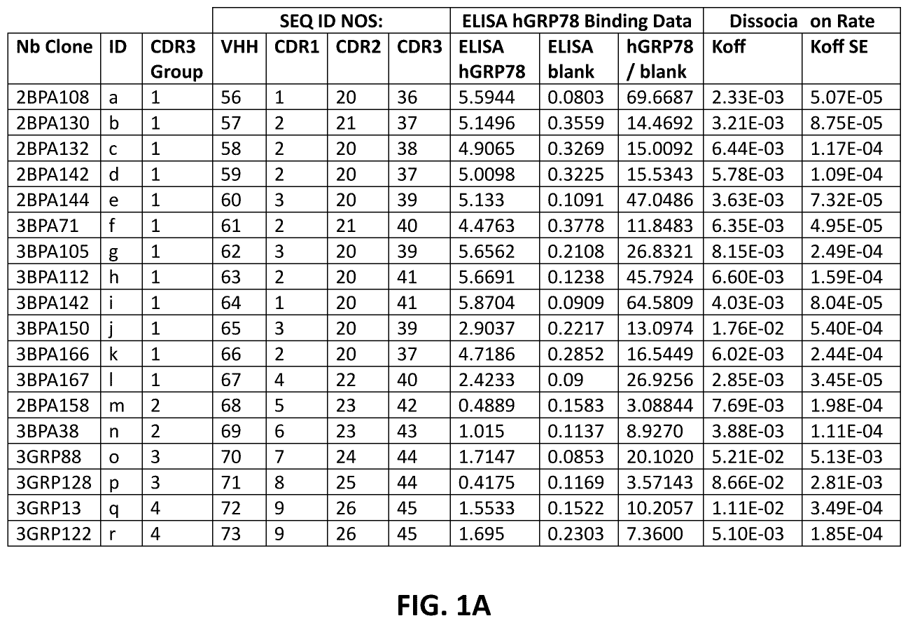

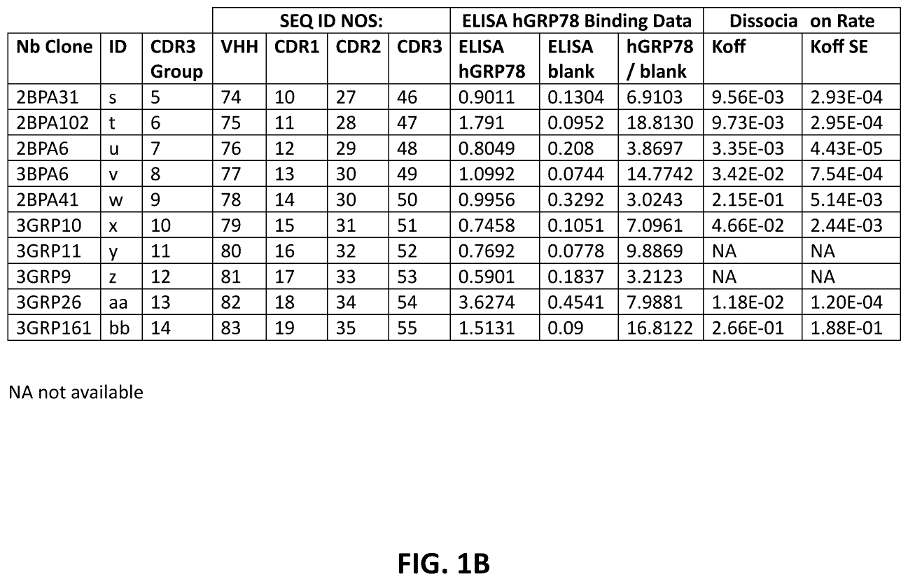

One aspect of the invention provides a protein such as an anti-huGRP78 nanobody or a fusion protein including an anti-huGRP78 nanobody amino acid sequence, the protein including:
(i) a nanobody amino acid sequence including the CDRs (CDR1, CDR2 and CDR3) of any of the anti-huGRP78 nanobodies disclosed herein, such as in FIG. 1A or FIG. 1B;
(ii) a nanobody amino acid sequence including the framework regions and the CDRs of any of the anti-huGRP78 nanobodies disclosed herein, such as in FIG. 1A or FIG. 1B; and/or
(iii) a nanobody amino acid sequence including the full nanobody amino acid sequence of any of the anti-huGRP78 nanobody sequences disclosed herein, i.e., any of SEQ ID NOS: 56-83.
   The nanobody or nanobody element of the protein may, for example, bind cell surface (cs) human GRP78.

The CDR sequences of the nanobody clones presented herein are delineated according to the IMGT numbering convention. The CDRs are surrounded by VHH domain framework regions (FRs) in the following manner: FR1 is the amino acid sequence preceding (N-terminal to) CDR1, FR2 is the amino acid sequence between CDR1 and CDR2, FR3 is the amino acid sequence between CDR2 and CDR3, and FR4 is the amino acid sequence following (C-terminal to) CDR3 to the end of the nanobody (VHH domain) sequence.

The protein may, for example, be a monomeric nanobody (VHH), a dimeric or multimeric nanobody wherein the nanobody components are the same or at least some are different and which may have the same or different binding specificities, or a fusion protein including a nanobody sequence and another protein sequence, such as a nanobody-Fc fusion protein or a nanobody-epitope tag fusion protein. Nanobody elements of a dimeric or multimeric nanobody or larger protein including such elements may, for example, be connected by a linker peptide, such as a (Gly₄Ser)₃ linker.

The protein may, for example, be used for the treatment of a cell surface GRP78-expressing cancer in a mammalian subject such as a human patient. The protein may, for example, be radiolabeled and/or drug-conjugated for use in the treatment of a cell surface GRP78-expressing cancer in a mammalian subject such as a human patient. The protein may, for example, be radiolabeled for use in detecting and/or imaging cell surface GRP78-expressing cancer cells and/or tumors in a mammalian subject such as a human patient.

The proteins and nanobodies disclosed herein may, for example, be linked directly or indirectly via a chemically conjugated chelator, to a radionuclide, for example, to target cytotoxic radiation to cell surface GRP78-expressing cells in mammalian subject such as a human patient, or to non-cytotoxically image cell surface GRP78-expression in a mammalian subject such as a human patient. For example, the antibody may be directly labeled with ¹³¹I according to the methods disclosed in U.S. Pat. No. 10,420,851 or the antibody may be chemically conjugated to a chelator, such as p-SCN-Bn-DOTA and labeled with a radionuclide such as ²²⁵Ac, according to the procedures described in U.S. Pat. No. 9,603,954.

The proteins and nanobodies may, for example, be linked to one or more cytotoxic drugs to target and deplete csGRP78-expressing cells in a mammalian subject such as a human patient. Thus, one aspect of the invention provides an antibody-drug-conjugate (ADC) that includes a nanobody according to the invention as a/the antibody component of the ADC.

The radionuclide may, for example, selected from ¹³⁴Ce, ⁴³Sc, ⁴⁴Sc, ⁴⁷Sc, ⁵⁵Co, ⁶⁰Cu, ⁶¹Cu, ⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ⁸⁹Zr, ⁹⁷Ru, ¹⁰⁵Rh, ¹⁰⁹Pd, ¹¹¹In, ^{117m}Sn, ¹⁴⁹Pm, ¹⁴⁹Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, and ²²⁷Th.

The chelator group in the various aspects of the invention may, for example, include: 1,4,7,10-tetraazacyclododecane-1,4,7-triacetic acid (DO3A) or a derivative thereof; 1,4,7-triazacyclononane-1,4-diacetic acid (NODA) or a derivative thereof; 1,4,7-triazacyclononane-1,4,7-triacetic acid (NOTA) or a derivative thereof; 1,4,7,10-tetraazacyclododecane-1,4,7,10-tetraacetic acid (DOTA) or a derivative thereof; 1,4,7-triazacyclononane, 1-glutaric acid-4,7-diacetic acid (NODAGA) or a derivative thereof; 1,4,7,10-tetraazacyclodecane, 1-glutaric acid-4,7,10-triacetic acid (DOTAGA) or a derivative thereof; 1,4,8,11-tetraazacyclotetradecane-1,4,8,11-tetraacetic acid (TETA) or a derivative thereof; 1,4,8,11-tetraazabicyclo[6.6.2]hexadecane-4,11-diacetic acid (CB-TE2A) or a derivative thereof; diethylene triamine pentaacetic acid (DTPA), its diester, or a derivative thereof; 2-cyclohexyl diethylene triamine pentaacetic acid (CHX-A″-DTPA) or a derivative thereof; deferoxamine (DFO) or a derivative thereof; 1,2-[[6-carboxypyridine-2-yl]methylamino]ethane (H2dedpa) or a derivative thereof; DADA or a derivative thereof; 1,4,7,10-Tetraazacyclododecane-1,4,7,10-tetra(methylene phosphonic acid) (DOTP) or a derivative thereof; 4-amino-6-[[16-[(6-carboxypyridine-2-yl)methyl]-1,4,10,13-tetraoxa-7,16-diazacyclooctadec-7-yl]methyl]pyridine-2-carboxylic acid (MACROPA-NH₂) or a derivative thereof; MACROPA or a derivative thereof; 1,4,7,10-tetrakis(carbamoylmethyl)-1,4,7,10-tetraazacyclododecane (TCMC) or a derivative thereof; {4-[2-(bis-carboxymethylamino)-ethyl]-7-carboxymethyl-[1,4,7]triazonin-1-yl}-acetic acid (NETA) or a derivative thereof; Diamsar or a derivative thereof; 1,4,7-triazacyclononane-1,4,7-tris[methyl (2-carboxyethyl)phosphinic acid (TRAP, PRP9, TRAP-Pr) or a derivative thereof; N,N′-bis(6-carboxy-2-pyridylmethyl)ethylenediamine-N,N′-diacetic acid (H4octapa) or a derivative thereof; N,N′-[1-benzyl-1,2,3-triazole-4-yl]methyl-N,N′-[6-(carboxy)pyridin-2-yl]-1,2-diaminoethane (H2azapa) or a derivative thereof; N,N″-[6-(carboxy)pyridin-2-yl]methyl]diethylenetriamine-N,N′,N″-triacetic acid (H5decapa) or a derivative thereof; N,N′-bis(2-hydroxy-5-sulfobenzyl)ethylenediamine-N,N′-diacetic acid (SHBED) or a derivative thereof; N,N′-bis(2-hydroxybenzyl)ethylenediamine-N,N′-diacetic acid (HBED) or a derivative thereof; 3,6,9,15-tetraazabicyclo[9.3.1]pentadeca-1 (15), 11,13-triene-3,6,9,-triacetic acid (PCTA) or a derivative thereof; desferrioxamine B (DFO) or a derivative thereof; N,N′-(methylenephosphonate)-N,N′-[6-(methoxycarbonyl)pyridin-2-yl]methyl-1,2-diaminoethane (H6phospa) or a derivative thereof; 1,4,7,10,13,16-hexaazacyclohexadecane-N,N′,N″,N″,N″″,N″″-hexaacetic acid (HEHA) or a derivative thereof; 1,4,7,10,13-pentaazacyclopentadecane-N,N′,N″,N′″,N″″-pentaacetic acid (PEPA) or a derivative thereof, or 3,4,3-LI(1,2-HOPO) or a derivative thereof.

Although throughout the present disclosure and claims various aspects or elements thereof are described in terms of “including” or “comprising,” corresponding aspects or elements thereof described in terms of “consisting essentially of” or “consisting of” are similarly disclosed. For example, while certain aspects of the invention have been described in terms of a method “including” or “comprising” administering a radiolabeled protein, corresponding methods instead reciting “consisting essentially of” or “consisting of” administering the radiolabeled protein are also within the scope of said aspects and provided by this disclosure.

In addition, compositions including an anti-GRP78 nanobody or protein including a GRP78-binding nanobody sequence, whether unlabeled, radiolabeled or drug-conjugated, may include one or more pharmaceutically acceptable carriers or pharmaceutically acceptable excipients. Such carriers are well known to those skilled in the art. For example, injectable drug delivery systems include solutions, suspensions, gels, microspheres and polymeric injectables, and can include excipients such as solubility-altering agents (e.g., ethanol, propylene glycol and sucrose) and polymers (e.g., polycaprolactones and PLGA's). An exemplary formulation may be as substantially described in U.S. Pat. No. 10,420,851 or International Pub. No. WO 2017/155937, incorporated by reference herein. For example, according to certain aspects, the formulation may include 0.5% to 5.0% (w/v) of an excipient selected from the group consisting of ascorbic acid, polyvinylpyrrolidone (PVP), human serum albumin (HSA), a water-soluble salt of HSA, and mixtures thereof. Certain formulations may include 0.5-5% ascorbic acid; 0.5-4% polyvinylpyrrolidone (PVP); and the nanobody or nanobody sequence-including protein in 50 mM PBS buffer, pH 7.

The nanobodies and nanobody sequence-including proteins disclosed herein may, for example, be unlabeled or labeled with a radionuclide, such as ¹³¹I or ²²⁵Ac, or conjugated to a cytotoxic drug, for use in the treatment of a cell surface GRP78-expressing solid cancer or hematological cancer, such as but not limited to testicular cancer, cervical cancer, glioma, esophageal cancer, ovarian cancer, gastric cancer, liver cancer, thyroid cancer, head & neck cancer, pancreatic cancer, uterine cancer, renal cancer, urothelial cancer, melanoma, colorectal cancer, lung cancer, small cell lung cancer (SCLC), non-small cell lung cancer (NSCLC), prostate cancer, castration-resistant metastatic prostate cancer (mCRPC), triple negative breast cancer (TNBC), breast cancer, hepatocellular carcinoma (HCC), cholangiocarcinoma, multiple myeloma, lymphoma, Hodgkin lymphoma, non-Hodgkin lymphoma, leukemia, acute myeloid leukemia (AML), or myelodysplastic syndrome (MDS). Solid cancers treated may be metastatic or non-metastatic.

### Example 1: Production of a Radiolabeled Anti-huGRP78 Nanobody

Radiolabeling: The nanobody may be chemically conjugated to a chelator-bearing bifunctional linker, such as a DOTA-bearing bifunctional linker, such as p-SCN-Bn-DOTA.

Chelation with a radionuclide, such as ¹⁷⁷Lu, ⁹⁰Y, or ²²⁵Ac may then be performed and efficiency and purity of the resulting radiolabeled protein, such as an anti-GRP78 nanobody, may be determined by HPLC and iTLC.

An exemplary labeling reaction for ²²⁵Ac is as follows: A reaction including 15 μl 0.15 M NH₄OAc buffer, pH=6.5 and 2 μL (10 μg) DOTA-anti-GRP78 (5 mg/ml) may be mixed in an Eppendorf reaction tube, and 4 μL ²²⁵Ac (10 μCi) in 0.05 M HCl subsequently added. The contents of the tube may be mixed with a pipette tip and the reaction mixture incubated at 37° C. for 90 min with shaking at 100 rpm. At the end of the incubation period, 3 μL of a 1 mM DTPA solution may be added to the reaction mixture and incubated at room temperature for 20 min to bind the unreacted ²²⁵Ac into the ²²⁵Ac-DTPA complex. Instant thin layer chromatography with 10 cm silica gel strip and 10 mM EDTA/normal saline mobile phase may be used to determine the radiochemical purity of 225Ac-DOTA-anti-GRP78 Nb through separating ²²⁵Ac-labeled anti-GRP78 (²²⁵Ac-DOTA-anti-GRP78 Nb) from free ²²⁵Ac (²²⁵Ac-DTPA). In this system, the radiolabeled nanobody stays at the point of application and ²²⁵Ac-DTPA moves with the solvent front. The strips may be cut in halves and counted in the gamma counter equipped with the multichannel analyzer using channels 72-110 for ²²⁵Ac to exclude its daughters.

Purification: The ²²⁵Ac-DOTA-nanobody may be purified either on PD10 columns pre-blocked with 1% HSA or using a centrifugal protein concentrator, such as a Pierce™ Protein Concentrator PES, 10K MWCO (Cat #88513; Thermo Fisher Scientific, Waltham, MA), with 2×1.5 mL washes, 3 min per spin. HPLC analyses of the ²²⁵Ac-DOTA-antibody after purification may be conducted using a Waters HPLC system equipped with flow-through Waters UV and Bioscan Radiation detectors, using a TSK3000SW XL column eluted with PBS at pH=7.4 and a flow rate of 1 ml/min.

Without limitation, the following aspects of the invention are provided by this disclosure:

Aspect 1. A protein including a human-GRP78 (huGRP78) binding, such as [human csGRP78]-binding, nanobody amino acid sequence which includes:
(i) the nanobody CDRs (CDR1, CDR2 and CDR3) of any of the nanobody clones set forth in FIG. 1A or FIG. 1B;
(ii) one or more of the framework regions (FR1, FR2, FR3 and FR4) and the CDRs of any of the nanobody clones set forth in FIG. 1A or FIG. 1B; and/or
(iii) the full nanobody sequence of any of the nanobody clones set forth in FIG. 1A or 1B, i.e., any of SEQ ID NOS: 56-83.

Aspect 2. The protein of aspect 1, including one or more of the following combinations (a through bb) of nanobody CDR amino acid sequences set forth in Table 1 below.

TABLE 1

| | | SEQ | | SEQ | | SEQ |
|---|---|---|---|---|---|---|
| | | ID | | ID | | ID |
| | CDR1 | NO: | CDR2 | NO: | CDR3 | NO: |
| a) | GHTSRTYA | 1 | IGWNGGT | 20 | AADDAKVLIVPHY | 36 |
| b) | GRTFRTYA | 2 | IGWNSGT | 21 | AADDSKVLIVPHY | 37 |
| c) | GRTFRTYA | 2 | IGWNGGT | 20 | AADDAKVLIVPHF | 38 |
| d) | GRTFRTYA | 2 | IGWNGGT | 20 | AADDSKVLIVPHY | 37 |
| e) | GRTFRTYS | 3 | IGWNGGT | 20 | AADDSKVLIVPVY | 39 |
| f) | GRTFRTYA | 2 | IGWNSGT | 21 | AADDSQVLIVPHY | 40 |
| g) | GRTFRTYS | 3 | IGWNGGT | 20 | AADDSKVLIVPVY | 39 |
| h) | GRTFRTYA | 2 | IGWNGGT | 20 | AADDAKVLIVPNY | 41 |
| i) | GHTSRTYA | 1 | IGWNGGT | 20 | AADDAKVLIVPNY | 41 |
| j) | GRTFRTYS | 3 | IGWNGGT | 20 | AADDSKVLIVPVY | 39 |
| k) | GRTFRTYA | 2 | IGWNGGT | 20 | AADDSKVLIVPHY | 37 |
| 1) | GRTFRIYA | 4 | IGWNAGT | 22 | AADDSQVLIVPHY | 40 |
| m) | TSFFSPSA | 5 | ITRGGTT | 23 | NLKSIDLQW | 42 |
| n) | ASFFSPSA | 6 | ITRGGTT | 23 | NLKSINLQW | 43 |
| o) | GFAFSSYY | 7 | INSGGGTT | 24 | VAGEKGTKDY | 44 |
| p) | GFTFSSYW | 8 | INRGGIIT | 25 | VAGEKGTKDY | 44 |
| q) | GFTFSRYG | 9 | ISWDGGTT | 26 | ATAPTTWGKQPYQ | 45 |
| | | | | | GPGYKT | |
| r) | GFTFSRYG | 9 | ISWDGGTT | 26 | ATAPTTWGKQPYQ | 45 |
| | | | | | GPGYKT | |
| s) | GFPFKHYW | 10 | VNNDGGGR | 27 | AATPRPRLGQGSD | 46 |
| | | | | | Y | |
| t) | GRTFSTYN | 11 | ISWSGNST | 28 | AKGWLVSDYDQVT | 47 |
| | | | | | Y | |
| u) | GRSFSTKT | 12 | ISSSGIT | 29 | AAHSSSRATITSP | 48 |
| | | | | | DY | |
| v) | GSVVSSNA | 13 | ITRSGLE | 30 | EKVDGMYA | 49 |
| w) | GRLYS | 14 | IVGSYGST | 30 | RTRGPNGDY | 50 |
| x) | NFLSSRFE | 15 | IFRDGNT | 31 | HVHILGRDY | 51 |
| y) | GFTFSSAT | 16 | ITDDGRDT | 32 | YADVTAGGPQRY | 52 |
| z) | GSIFKVAG | 17 | ITSGGGT | 33 | SAQGADSNYALFR | 53 |
| | | | | | S | |
| aa) | GFTFSNYA | 18 | ITNDGLRT | 34 | GVRGTSRWGRSFR | 54 |
| | | | | | S | |
| bb) | GNIFSPNA | 19 | ITSSGLT | 35 | KWAVFRDYGMGAD | 55 |
| | | | | | DY | |

Aspect 3. The protein of aspect 1 or 2, wherein said protein is a nanobody.

Aspect 4. The protein of aspect 1 or 2, wherein said protein includes more than one nanobody portion.

Aspect 5. A pharmaceutical composition including the protein of any one of the preceding aspects and at least one pharmaceutically acceptable excipient.

Aspect 6. A radiopharmaceutical composition including the protein of any one of aspects 1-4 linked to a radionuclide.

Aspect 7. The radiopharmaceutical composition of aspect 6, further including at least one pharmaceutically acceptable excipient.

Aspect 8. The radiopharmaceutical composition of aspect 6 or 7, wherein the radionuclide is an alpha particle emitter.

Aspect 9. The radiopharmaceutical composition of aspect 6 or 7, wherein the radionuclide is a beta particle emitter.

Aspect 10. The radiopharmaceutical composition of aspect 6 or 7, wherein the radionuclide includes ¹³¹I.

Aspect 11. The radiopharmaceutical composition of aspect 6 or 7, wherein the radionuclide includes ²²⁵Ac, ¹⁷⁷Lu or ⁹⁰Y.

Aspect 12. A composition including the protein of any one of aspect 1-4, chemically conjugated to a chelator.

Aspect 13. The composition of aspect 12, further including a radionuclide chelated by the chelator.

Aspect 14. The composition of aspect 12 or 13, wherein the chelator includes DOTA or a DOTA derivative.

Aspect 15. The composition of aspect 14, wherein further including ¹⁷⁷Lu, ⁹⁰Y or ²²⁵Ac chelated by the DOTA or DOTA derivative.

Aspect 16. The composition of any one of aspects 12-15, further including at least one pharmaceutically acceptable excipient.

Aspect 17. A method for treating a hematological or solid cancer in a mammalian subject, including administering to the subject a therapeutically effective amount of the protein, composition or radiopharmaceutical composition of any one of the preceding aspects.

Aspect 18. The method of aspect 18, wherein the hematological or solid cancer expresses or overexpresses cell surface GRP78.

Aspect 19. The method of aspect 17 or 18, wherein the mammalian subject is human.

Aspect 20. A method for diagnosing a mammalian subject, such as a human, with a cell surface GRP78-expressing cancer, including:
administering to the subject a radiolabeled form of a protein according to any one of aspects 1-4; and
after a time sufficient time from the administration for the radiolabeled form of the protein to bind csGRP78 that may be present within the tissues of the subject, detecting/imaging the radiation emitted by the radiolabeled protein-bound GRP78 in the subject.

Aspect 21. Use of a therapeutically effective amount of the protein, composition or radiopharmaceutical composition of any one of aspects 1-16 for the treatment of a hematological or solid cancer in a mammalian subject, such as a cell surface GRP78-expressing hematological or solid cancer.

Aspect 22. The use of aspect 21, wherein the subject is human.

Aspect 21. Use of a radiolabeled form of the protein of any one of aspects 1-4, for the diagnosis of a hematological or solid cancer in a mammalian subject, such as a cell surface GRP78 expressing hematological or solid cancer.

Aspect 22. The use of aspect 21, wherein the subject is human.

Aspect 23. Use of a protein according to any one of aspects 1˜4 in the preparation of a medicament for the treatment of a hematological or solid cancer in a mammalian subject, such as a cell surface GRP78 expressing hematological or solid cancer.

Aspect 24. The use of aspect 23, wherein the subject is human.

Aspect 25. Use of a protein according to any one of aspects 1˜4 in the preparation of a radiolabeled diagnostic imaging agent for the detection of cell surface GRP78-expressing hematological or solid cancer in a mammalian subject.

Aspect 26. The use of aspect 25, wherein the subject is human.

Aspect 27. The protein or composition of any one of aspects 1-16 for the treatment of a hematological or solid cancer, such as a cell surface GRP78 expressing hematological or solid cancer, in a mammalian subject, such as a human.

Aspect 28. The protein, composition, method or use of any one of the preceding aspects wherein the protein includes one or more of the nanobody amino acid sequences:

a)
(SEQ ID NO: 56)
QVQLQEAGGGLVQAGGSLRLSCAASGHTSRTYAMGWFRQAPGKEREFVAR
IGWNGGTYYADFVKGRFTISRDGAKNTVYLQMNSLKPEDTAVYYCAADDA
KVLIVPHYXGQGTQVTVSS;
b)
(SEQ ID NO: 57)
QVQLQESGGGLVQAGGSLRLSCAASGRTFRTYAMGWFRQAPGKEREFVAR
IGWNSGTYYADSVKGRFTISRDGAKNTVYLQMNSLKPEDTAVYYCAADDS
KVLIVPHYWGQGTQVTVSS;
c)
(SEQ ID NO: 58)
QVQLQESGGGLVQAGGSLRLSCAASGRTFRTYAMGWFRQAPGKEREFVAR
IGWNGGTYYADSVKGRFTISRDGAENTVYLQMNSLKPEDTAVYYCAADDA
KVLIVPHFWGQGTQVTVSS;
d)
(SEQ ID NO: 59)
QVQLQESGGGLVQAGGSLRLSCAASGRTFRTYAMGWFRQAPGKEREFVAR
IGWNGGTYYVDSVKGRFTISRDGAKNTVYLQMNSLKPEDTAVYYCAADDS
KVLIVPHYWGQGTQVTVSS;
e)
(SEQ ID NO: 60)
QVQLQESGGGLVQPGDSLSLSCAASGRTFRTYSMGWFRQAPGKEREFVAR
IGWNGGTYYADSVKGRFTISRDGAKNTVYLQMNSLRPEDTAIYYCAADDS
KVLIVPVYWGQGTQVTVSS;
f)
(SEQ ID NO: 61)
QVQLQESGGGLVQAGGSLRLSCAASGRTFRTYAMGWFRQAPGKEREFVAR
IGWNSGTHYADSVKGRFTISRDGAKNTVYLQMNSLTPEDTAVYYCAADDS
QVLIVPHYWGQGTQVTVSS;
g)
(SEQ ID NO: 62)
QVQLQESGGGLVQAGGSLRLSCAASGRTFRTYSMGWFRQAPGKEREFVAR
IGWNGGTYYADSVKGRFTISRDGAKNTVYLQMNSLRPEDTAIYYCAADDS
KVLIVPVYWGQGTQVTVSS;
h)
(SEQ ID NO: 63)
QVQLQESGGGLVQAGGSLRLSCTASGRTFRTYAMGWFRQAPGKEREFVAR
IGWNGGTYYTDSVKGRFTISRDGAKNTVWLQMDSLKPEDTAVYYCAADDA
KVLIVPNYWGQGTQVTVSS;
i)
(SEQ ID NO: 64)
QVQLQESGGGLVQAGDSLRLSCAVSGHTSRTYAMGXFRQAPGKEREFVAR
IGWNGGTYYADFVKGRFTISRDGAKNLVWLQMNSLKPEDTAVYYCAADDA
KVLIVPNYWGQGTQVTVSS;
j)
(SEQ ID NO: 65)
QVQLVESGGGLVQAGGSLRLSCAASGRTFRTYSMGWFRQAPGKEREFVAR
IGWNGGTYYADSVKGRFTISRDGAKNTVYLQMNSLRPEDTAIYYCAADDS
KVLIVPVYWGQGTQVTVSS;
k)
(SEQ ID NO: 66)
QVQLQESGGGLVQAGGSLRLSCAASGRTFRTYAMGWFRQAPGKEREFVAR
IGWNGGTYYADSVKGRFTISRDGAKNTVYLQMNSLKPEDTAVYYCAADDS
KVLIVPHYWGQGTQVTVSS;
l)
(SEQ ID NO: 67)
QVQLQESGGGLVQAGGSLRLSCAASGRTFRIYAMGWFRQAPGKEREFVAR
IGWNAGTYVADSVKGRFTISRDGTKNTVYLQMNSLKPEDTAVYYCAADDS
QVLIVPHYWGQGTQVTVSS;
m)
(SEQ ID NO: 68)
QVQLQESGGGLVQAGGSLRLSCAASTSFFSPSAMGWYRQAPGKQRELVAT
ITRGGTTNYADSVKGRFTISKDNADNTVFLQMNSLKPDDTAVYYCNLKSI
DLQWWGQGTQVTVSS;
n)
(SEQ ID NO: 69)
QVQLQESGGGLVQAGGSLRLSCAASASFFSPSAMGWYRQAPGKQRELVAI
ITRGGTTNYADSVKGRFTISKNNADNTVFLQMNSLKPDDTAVYYCNLKSI
NLQWWGQGTQVTVSS;
o)
(SEQ ID NO: 70)
QVQLQESGGGNVQPGGSLRLSCAASGFAFSSYYMYWVRQAPGKGLEWVSI
INSGGGTTTYADSVKGRFTISRDNAKNTLYLQMNSLKPEDTAVYYCVAGE
KGTKDYWGQGTQVTVSS;
p)
(SEQ ID NO: 71)
QVQLQESGGGVVQPGGSLRLSCAASGFTFSSYWMYWVRQTPGKGLEWVSL
INRGGIITYYSDSVKGRFTISRDNAKNTVYLQMNSLKPEDTAVYYCVAGE
KGTKDYWGQGTQVTVSS;
q)
(SEQ ID NO: 72)
QVQLQESGGGLVQAGGSLRLSCAASGFTFSRYGMVWFRQSPGKEREIVAA
ISWDGGTTYYADSVKGRFTISRHNAKDTVDLQMNSLNPEDTAVYYCATAP
TTWGKQPYQGPGYKTWGQGTQVTVSS;
r)
(SEQ ID NO: 73)
QVQLQESGGGLVQAGGSLRLSCAASGFTFSRYGMVWFRQSPGKERELVSA
ISWDGGTTYYVDSVKGRFTISRDNAVDTVYLQMNSLKPEDTAVYYCATAP
TTWGKQPYQGPGYKTWGQGTQVTVSS;
s)
(SEQ ID NO: 74)
QVQLQESGGGLVXPGGSLRLSCVTSGFPFKHYWMYWVRQAPGKGVEWVSI
VNNDGGGRHYSDSVKGRFTISRDNDKNVLYLEMNNLKPEDTARYYCAATP
RPRLGQGSDYWGQGTQVTVSS;
t)
(SEQ ID NO: 75)
QVQLQESGGGLVQAGGSLRLSCAASGRTFSTYNMAWFRQAPEKEREIVAV
ISWSGNSTYYADSVKGRFTISRDNAKNTVYLQMNSLKPEDTAVYYCAKGW
LVSDYDQVTYWGQGTQVTVSS;
u)
(SEQ ID NO: 76)
QVQLQESGGGLVQAGGSLRLSCAASGRSFSTKTMGWFRQAPGKERELVAA
ISSSGITYYADSMKGRFTISRDNARNMVFLQMNNLNPEDTAVYFCAAHSS
SRATITSPDYWGQGTQVTVSS;
v)
(SEQ ID NO: 77)
QVQLQESGGGSVQAGGSLRLSCVVSGSVVSSNAMGWYGQAPGKQRELVAW
ITRSGLENYKDSVRGRFTISRDNAKNTAYLQMDNLRPEDTAVYYCEKVDG
MYARGQGTQVTVSS;
w)
(SEQ ID NO: 78)
QVQLQESGGGLVQAGGSLRLSCAPSGRLYSVAWFRQRSGKEREFVSSIVG
SYGSTFYADSVKGRFTISRDNAKNMLYLQMDSLKPEDTAVYYCRTRGPNG
DYWGQGTQVTVSS;
x)
(SEQ ID NO: 79)
QVQLQESGGGLVQAGESLRLSCAGTNFLSSRFEMGWYRQIPGEXRELVAR
IFRDGNTDYTDSVRGRFTISRDTAKNTIDLQMNNLKPEDSAGYLCHVHIL
GRDYWGQGTQVTVSS;
y)
(SEQ ID NO: 80)
QVQLQESGGGLVQPGGSLRLSCATSGFTFSSATMIWVRQAPGKGPEWVAI
ITDDGRDTAYAASVQGRFTIARDNAKNMLYLQMNSLKSDDTAVYQCYADV
TAGGPQRYWGQGTQVTVSS;
z)
(SEQ ID NO: 81)
QVQLQESGGGLVQSGGSLRLSCAVSGSIFKVAGMQWYRQAPEKQRELVAS
ITSGGGTNYADSVKGRFTISRDNAKNTAYLQMNSLTPEDTAVYFCSAQGA
DSNYALFRSRGQGTQVTVSS;
aa)
(SEQ ID NO: 82)
QVQLQESGGGLVQPGGSLRLSCTASGFTFSNYAMDWVRQAPGKGLEWVAA
ITNDGLRTEYEDTVQGRFTISRDNAKNTLYLQMNSLKPEDTAVYYCGVRG
TSRWGRSFRSWGQGTQVTVSS;
and
bb)
(SEQ ID NO: 83)
QVQLQESGGGLVQPGGSLRLSCVASGNIFSPNAVGWYRQAPGKQRESVAD
ITSSGLTRYADDVKGRFVISRDNAKGTVDLQMNSLKPEDTAVYYCKWAVF
RDYGMGADDYWGQGTQVTVSS;

wherein X is a Q (glutamine) or W (tryptophan) residue.

Aspect 29. The protein, composition, method or use of any one of the preceding aspects wherein the 1 protein further includes N-terminal amino acid sequence AAAYPYDVPDYGSHHHHHH (SEQ ID NO: 84), for example, fused immediately to the N-terminus of FR4 of the nanobody. This sequence is a combined hemagglutinin (HA) and Histidine (His) 6 epitope tag (SEQ ID NO:87).

Aspect 30. The protein, composition, method or use of any one of the preceding aspects wherein the protein is a nanobody-Fc fusion protein.

Aspect 31. The protein, composition, method or use of aspect 30, wherein the Fc portion of the nanobody-Fc fusion protein is a non-camelid Fc region, such as a mouse Fc region or a human or human-derived Fc region.

Aspect 32. The protein, composition, method or use of aspect 30 or 32, wherein the Fc portion is connected to the nanobody (VHH) portion via a linker peptide (disposed as the N-terminal end of the nanobody sequence (so that the amino-to-carboxyl arrangement of elements is nanobody-linker-Fc).

Aspect 33. The protein, composition, method or use of aspect 30 or 32, wherein the linker peptide includes the sequence STMVRS (SEQ ID NO: 85), EPKSCDKTHTCPPCP (SEQ ID NO: 88; derived from human IgG1 hinge region), or VPRDCGCKPCICT (SEQ ID NO:90; derived from mouse IgG1 hinge region).

Aspect 34. The protein, composition, method or use of any one of aspects 30-33, wherein the Fc region includes the sequence:

(human Fc gamma1 type with N-terminal hinge
region)
SEQ ID NO: 86
DKTHTCPPCPAPELLGGPSVFLFPPKPKDTLMISRTPEVTCVVVDVSHED
PEVKFNWYVDGVEVHNAKTICPREEQYNSTYRVVSVLTVLHQDWLNGICE
YKCKVSNICALPAPIEKTISICAKGOPREPOVYTLPPSREEMTICNOVSL
TCLVKGFYPSDIAVEWESNGOPENNYKTITTVLDSDGSFFLYSICLTVDK
SRWQQGNVFSCSVMHEALHNHYTQKSLSLSPGK;;
(human Fc gamma1 type)
SEQ ID NO: 89
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHE
ALHNHYTQKSLSLSPGK;;
or
(mouse Fc gamma1 type)
SEQ ID NO: 91
VPEVSSVFIFPPKPKDVLTITLTPKVTCVVVDISKDDPEVQFSWFVDDVE
VHTAQTQPREEQFNSTFRSVSELPIMHQDWLNGKEFKCRVNSAAFPAPIE
KTISKTKGRPKAPQVYTIPPPKEQMAKDKVSLTCMITDFFPEDITVEWQW
NGQPAENYKNTQPIMNTNGSYFVYSKLNVQKSNWEAGNTFTCSVLHEGLH
NHHTEKSLSHSPGK;.

Suitable pairings of linker sequences and Fc region sequences include, for example, SEQ ID NO: 85 with SEQ ID NO:86, SEQ ID NO:88 with SEQ ID NO:89, and SEQ ID NO:90 with SEQ ID NO:91, for nanobody Fc fusion proteins including the structure VHH-linker-Fc.

### Methods

The following methods were used to generate and characterize the anti-huGRP78 nanobodies of this disclosure.

### Generation and Identification of Anti-huGRP78 Nanobodies

### Immunizations

Two llamas were subcutaneously injected, each time and per animal with about 150 μg of recombinant huGRP78 carrying an N-terminal His6 tag (Novus Biologicals LLC a Bio-Techne Brand, Centennial, CO, USA, Cat. No. NBC1-18378). The adjuvant used was Gerbu adjuvant P. The interval between the first two injections was one week, while the intervals between all other injections were two weeks. Four and eight days post last injection, about 100 ml anticoagulated blood was collected from each animal for lymphocyte preparation. Animal health was regularly monitored. None of the animals showed any signs of discomfort during the whole immunization period.

### Construction of Two Independent VHH Libraries

Individual VHH libraries were constructed from each llama's lymphocytes to screen for the presence of antigen-specific nanobodies. To this end, a mix (ratio of 1:1) of total RNA from peripheral blood lymphocytes from 4 d.p.i. and 8 d.p.i. was used as template for first strand cDNA synthesis with an oligo(dT) primer. Using this cDNA, the VHH encoding sequences were amplified by PCR. For each llama, PCR fragments were digested with SapI, and cloned into the SapI site of the phagemid vector pMECS-GG. The nanobody gene cloned in pMECS-GG vector contains PelB signal sequence at the N-terminus and HA tag and His6 tag at the C-terminus (PelB leader-nanobody-HA-His6). The PelB leader sequence directs the nanobody to the periplasmic space of the E. coli and the HA and His6 tags can be used for the purification and detection of nanobody (e.g., in ELISA, Western Blot, etc.). In pMECS-GG vector, the His6 tag is followed by an amber stop codon (TAG) and this amber stop codon is followed by gene III of M13 phage. In suppressor E. coli strains (e.g., TG1), the amber stop codon is read as glutamine and therefore the nanobody is expressed as a fusion protein with protein III of the phage which results in the display of nanobody on the phage coat for panning. In TG1 suppressor strains, the efficiency of suppression is not 100% and therefore the expression of nanobodies in suppressor strains leads to two different types of nanobody molecules, fused to protein III and without protein III). In non-suppressor E. coli strains (e. g., WK6), the amber stop codon is read as a stop and therefore the resulting nanobody is not fused to protein III. Thus, nanobodies cloned in pMECS-GG vector can be expressed in the periplasmic space by transforming a non-suppressor strain (e.g., WK6) with this plasmid.

The VHH library obtained from the first animal was designated Core 180. The Core 180 library consists of about 3×108 independent transformants, with about 94% of transformants harboring the vector with the right insert size of VHH-encoding sequences. The library obtained from the second animal, Core 181, consists of about 4×108 independent transformants, with about 94% of transformants harboring the vector with the right insert size.

Isolation of huGRP78-Specific Nanobodies

Generated phage libraries were separately panned on solid-phase coated recombinant huGRP78 (100 μg/ml in 100 mM NaHCO3 pH 8.2) for 3 rounds. The enrichment for antigen-specific phages was assessed after each round of panning by comparing the number of phagemid particles eluted from antigen-coated wells with the number of phagemid particles eluted from negative control (uncoated blocked) wells.

For Core 180, these experiments suggested that the phage population was enriched for antigen-specific phages about 2-fold, 10-fold and 60-fold after the 1st, 2nd and 3rd round, respectively. In total, 380 colonies (190 from round 2, 190 from round 3) were randomly selected and analyzed by ELISA for the presence of antigen-specific nanobodies in their periplasmic extracts (ELISA using crude periplasmic extracts including soluble nanobodies). The antigen used for panning and for ELISA screening was the same one as used for immunization, except that the batches were different, using uncoated blocked wells as negative controls (blank). Out of the 380 colonies tested by ELISA, 126 colonies scored positive for huGRP78. Based on sequence data of the 126 positive colonies, 9 different nanobodies were identified, belonging to 7 different CDR3 groups (B-cell lineages). Nanobodies belonging to the same CDR3 group (same B-cell lineage) are very similar and their amino acid sequences suggest that they are from clonally-related B-cells resulting from somatic hypermutation or from the same B-cell but diversified due to RT and/or PCR error during library construction. Nanobodies belonging to the same CDR3 group recognize the same epitope but their other characteristics (e.g. affinity, potency, stability, expression yield, etc.) can be different. Nanobodies resulting from the panning/ELISA screening of the Core 180 library bear “GRP” in their names.

When panning the Core 181 library on huGRP78, the enrichment experiments suggested that the phage population was enriched for antigen-specific phages about 3-fold, 16-fold and 100-fold after the 1st, 2nd and 3rd round, respectively. Here also, in total 380 colonies (190 from round 2, 190 from round 3) were randomly selected and analyzed by ELISA for the presence of antigen-specific nanobodies in their periplasmic extracts, as described above. Out of the 380 colonies tested by ELISA, 225 colonies scored positive for huGRP78. Based on sequence data of the 225 positive colonies, 19 different nanobodies were identified, belonging to 7 different CDR3 groups (B-cell lineages). Nanobodies resulting from the panning/ELISA screening of the Core 181 library bear “BPA” in their names.

In summary, twenty-eight (28) unique human GRP78-specific nanobodies belonging to fourteen (14) different CDR3 groups were identified. The ELISA huGRP78 binding data for the nanobody clones is shown in FIGS. 1A and 1B. Clones 2BPA31, 2BPA108, and 3GRP10 have an internal Amber stop codon (TAG). In suppressor E. coli strains such as TG1 used here, the TAG codon is read as glutamine (Q). However, in non-suppressor strains such as WK6, the TAG is read as a stop codon resulting in truncated (non-functional) nanobodies. Therefore, care must be taken when expressing these TAG-containing nanobodies in non-suppressor E. coli strains such as WK6, so as to replace the Amber stop codon by a codon for glutamine (Q) during the cloning. For clone 2BPA108, the consensus replacement for this position is a codon for tryptophan (W). Clone 3BPA142 has an internal Opal stop codon (TGA), which may result in a truncated protein, although E. coli seems to have a low percentage of read-through for the opal codon. The expected amino acid in this position for this clone is tryptophan (W).

### Off-Rate Ranking of Identified Anti-huGRP78 Nanobodies

Off-rates on biotinylated recombinant human GRP78 were determined and ranked for the 28 anti-human GRP78 nanobodies, as follows.

Periplasmic extracts (PEs) of the nanobodies were prepared for use in the off-rate ranking experiments.

Human GRP78 coated Octet Streptavidin (SA) tips were prepared for use in the experiments as follows. The recombinant human GRP78 was first biotinylated (biotin/protein ratio 1:1) and then immobilized on SA-coated tips at 5 μg/ml in PBS. Human GRP78 was allowed to bind to SA tips for 300s yielding about 3.5 nm units of immobilized proteins, as compared with baseline. These conditions were used for ranking the off-rates of the nanobodies.

Off-rates were determined as follows. Per Nb, 200 μl of PE was mixed with 2 μl of 10% Tween20-PBS in a well of a 96 well black-plate to reduce aspecific interactions. For each set of experiments, PE from an E. coli containing the empty pMECS-GG vector was used as negative control. This plate was loaded into a Fortebio Octet Red BLI instrument (Forte Biosciences, Inc., Dallas, TX, USA) and brought into contact with antigen-coated Octet tips. Consequently, the binding profile for each clone was determined. Using the Fortebio Data Analysis Software, the blanks were subtracted and the curves were aligned. Based on these curves, the off-rates were calculated using a 1:1 binding model.

The SA tips, coated as described above with human GRP78, were allowed to interact with PEs of anti-human GRP78 specific Nbs. After contact with the PEs, the tips were put into wells containing only buffer to measure dissociation. Based on these values, the off-rates were determined for clones that demonstrated detectable binding (see k-off data for the nanobody clones in FIGS. 1A and 1B).

More than the half of the nanobodies tested in this off-rate ranking experiment showed reliable binding responses (between about 0.1 and 0.5 nm) in the association step, proving that the amount of nanobody present in the periplasmic extracts was sufficient to saturate the target protein and hence yield reliable results. The great majority of the good binders, belong to CDR3 group 1, the largest CDR3 group. All the members of this group have a high and reliable binding response, with a quite strong k-off value (between 2-8×10-3 1/s), with the only exception of clone 3BPA150, the weakest clone of this group, with a k-off of about 1×10-2 1/s. Outside of CDR3 group 1 the best ranked binders are clone 3BPA38 from CDR3 group number 2, 3GRP122 from CDR3 group number 4 and 2BPA6 from CDR3 group number 7.

### Epitope Binning of Ten Anti-huGRP78 Nanobodies

Epitope binning was performed for 10 selected anti-huGRP78 nanobodies (belonging to 10 different CDR3 groups) on human GRP78. The nanobody clones were selected based on the results of the off-rate ranking experiment previously described. These epitope binning experiments were performed by bio-layer interferometry (BLI) using a ForteBio Octet system.

Periplasmic extracts of the selected nanobodies were used in the binning experiments.

Human GRP78 antigen was biotinylated at non-saturating conditions (ratio 1:1) and then used at 10 μg/ml in the “Ligand scouting immobilization conditions” protocol for SA Biosensors as detailed in Fortebio Technical Note 26, in conjunction with a Fortebio Octet Red instrument.

The binning assay was performed as follows. The target antigen was coated on a series of Octet SA tips. Each series of tips was first soaked in a periplasmic extract containing one specific nanobody (termed first Nb). After the response signals reached saturation (where possible with extracts), the tips were transferred to a mix of PEs containing again the first Nb (at the same concentration) plus a different Nb (termed secondary Nb) for each tip of the series. Secondary Nbs that still bind the target antigen in the presence of the first Nb will show an increased response, as compared to the binding response of the first Nb alone. As a control, one tip was also incubated with the first Nb in the second incubation step, which should not lead to an increased response. This was repeated for each Nb as primary Nb until all Nbs were tested against each other in both directions (as primary and as secondary binder). From the response data, the bins were deduced for each Nb.

The biotinylated human GRP78 antigen was coated onto Octet SA tips. Ten specific Nbs from 10 different CDR3 groups (namely, 3GRP10, 3GRP26, 3GRP88, 3GRP122, 2BPA6, 2BPA31, 2BPA102, 3BPA6, 3BPA38 and 3BPA167) were tested. These clones were selected based on the k-off values obtained after the off-rate ranking experiment, choosing the best clone of each multi-clones CDR3 group.

All the PEs used were able to saturate the immobilized huGRP78 in the first binding step (showing a high binding response between about 0.2 and 0.9 nm). This was also confirmed by the low responses seen in our internal controls, i.e. when all the Nbs were paired with themselves for the second step of the assay. The responses were calculated for each primary Nb and for the subsequent mix with the secondary Nb. Then, the percentage of additional binding of the secondary versus the primary step was calculated. The value of the internal control was always subtracted from the results.

Data analysis identified three epitope bins. Every clone belongs to interdependent bins, since they block or they are blocked, only when they are tested as first or second nanobody:

Clones 3GRP26 (bin 1) and 3BPA167 (bin 3) have the highest binding response in the first association step, and do not seem to interfere with the binding of each other when clone 3GRP26 is used as first Nb. Moreover, they both totally block all the other clones in the second association step but, at the same time, they always show high binding when used as second Nb.

Clones 3GRP88, 2BPA31, 3GRP122, 2BPA6, 3BPA38, 3GRP10, 2BPA102 and 3BPA6 all share bin 2, because they behave in a similar way when they are tested against the other clones: they are able to block the binding of the other Nbs (except for the Nbs belonging to bins 1 and 3) when tested as first Nb, but they are also mostly blocked by all the other clones, when tested as second Nb. Because of slight differences in extra binding observed within bin 2, three sub-bins were defined: 2a, 2b and 2c. A slightly increased binding signal is observed for most of the clones in bin 2a and 2b versus clones from 2c as first Nb.

In summary, for the 10 anti-human GRP78 Nbs tested, there are a total of 3 epitope bins, with bin 2 consisting of three sub-bins (2a, 2b, and 2c), as follows.
Bin 1: 3GRP26;
Bin 2a: 3GRP88 and 2BPA31;
Bin 2b: 3GRP122, 2BPA6 and 3BPA38;
Bin 2c: 3GRP10, 2BPA102 and 3BPA6; and
Bin 3: 3BPA167.

Clones within the same bin recognize the same or overlapping epitopes. Independent bins (not blocked in any direction by another bin's Nbs) have distinct and independent epitopes. However, when the binding of one Nb only partially interferes with the binding of the second Nb (or in just one direction of the assay), the epitope of the second Nb can either partially overlap/obstruct the epitope of the first Nb or the binding of one of the Nbs induces a conformational shift in the antigen that distorts or makes inaccessible the epitope of the other Nb. Given the tendency of Nbs to bind to structural, rather than linear, epitopes any change in conformation could have drastic effects on the availability of the epitope. Consequently, for non-independent bins, no definite statements can be made about the independence of the epitopes in question. For this set of clones, every bin (and clone) shows some form of interdependency.

### HL60 Flow Cytometric Nanobody Cell Binding Assays

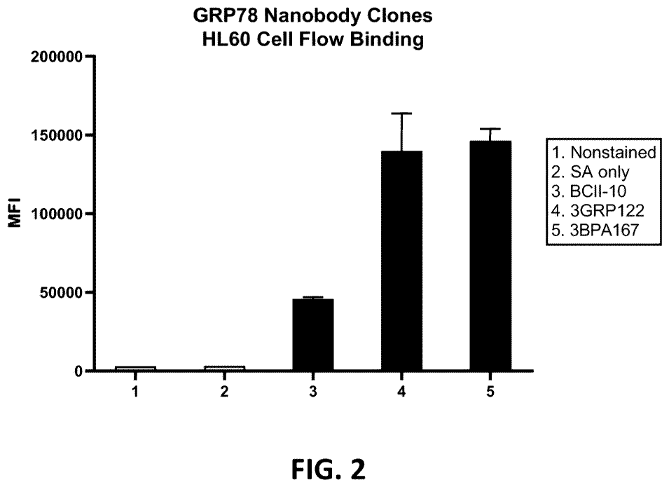

The cell binding capabilities of the anti-huGRP78 nanobodies 3GRP122 and 3BPA167 were evaluated using flow cytometry, as follows. Human acute myeloid leukemia (AML) model HL60 cells were plated at 1×10⁵ cells per well and anti-huGRP78 nanobodies or a negative control nanobody (nanobody BCII-10) were added at a concentration of 125 μg/mL or 400 μg/mL and incubated for one hour at 4° C., followed by a wash. The Alexa Fluor 647-conjugated anti-Alpaca IgG VHH domain secondary antibody was then added to the cells (1:500 dilution) and incubated for thirty to forty-five minutes at 4° C., followed by a wash. Cell binding was then detected by measuring the cell staining signal with a BD Accuri C6 flow cytometer (Becton Dickinson, Franklin Lakes, NJ, USA) and data analyzed using GraphPad Prism software (Dotmatics LLC, Boston, MA, USA). Data are shown in FIG. 2 where “Nonstained” indicates a cells-only control (no labeling agents used), and “SA” refers to a secondary antibody-only control (no primary antibody/nanobody used). The results show that nanobodies 3GRP122 and 3BPA167 each specifically bind HL60 cells versus the controls.

While various aspects and embodiments have been illustrated and described herein, it will be appreciated that various changes can be made without departing from the spirit and scope of the invention(s). Moreover, features described in connection with one aspect of the invention may be used in conjunction with other aspects of the invention, even if not explicitly exemplified in combination within.

1. A protein comprising a huGRP78-binding nanobody amino acid sequence, comprising one or more of the following combinations of nanobody CDR1, CDR2 and CDR3 amino acid sequences:

| | | | |
|---|---|---|---|
| i.) | SEQ ID NO: 1, | SEQ ID NO: 20, and | SEQ ID NO: 36; |
| ii.) | SEQ ID NO: 2, | SEQ ID NO: 21, and | SEQ ID NO: 37; |
| iii.) | SEQ ID NO: 2, | SEQ ID NO: 20, and | SEQ ID NO: 38; |
| iv.) | SEQ ID NO: 2, | SEQ ID NO: 20, and | SEQ ID NO: 37; |
| v.) | SEQ ID NO: 3, | SEQ ID NO: 20, and | SEQ ID NO: 39; |
| vi.) | SEQ ID NO: 2, | SEQ ID NO: 21, and | SEQ ID NO: 40; |
| vii.) | SEQ ID NO: 3, | SEQ ID NO: 20, and | SEQ ID NO: 39; |
| viii.) | SEQ ID NO: 2, | SEQ ID NO: 20, and | SEQ ID NO: 41; |
| ix.) | SEQ ID NO: 1, | SEQ ID NO: 20, and | SEQ ID NO: 41; |
| x.) | SEQ ID NO: 3, | SEQ ID NO: 20, and | SEQ ID NO: 39; |
| xi.) | SEQ ID NO: 2, | SEQ ID NO: 20, and | SEQ ID NO: 37; |
| xii.) | SEQ ID NO: 4, | SEQ ID NO: 22, and | SEQ ID NO: 40 |
| xiii.) | SEQ ID NO: 5, | SEQ ID NO: 23, and | SEQ ID NO: 42; |
| xiv.) | SEQ ID NO: 6, | SEQ ID NO: 23, and | SEQ ID NO: 43; |
| xv.) | SEQ ID NO: 7, | SEQ ID NO: 24, and | SEQ ID NO: 44; |
| xvi.) | SEQ ID NO: 8, | SEQ ID NO: 25, and | SEQ ID NO: 44; |
| xvii.) | SEQ ID NO: 9, | SEQ ID NO: 26, and | SEQ ID NO: 45; |
| xviii.) | SEQ ID NO: 10, | SEQ ID NO: 27, and | SEQ ID NO: 46; |
| xix.) | SEQ ID NO: 11, | SEQ ID NO: 28, and | SEQ ID NO: 47; |
| xx.) | SEQ ID NO: 12, | SEQ ID NO: 29, and | SEQ ID NO: 48; |
| xxi.) | SEQ ID NO: 13, | SEQ ID NO: 30, and | SEQ ID NO: 49; |
| xxii.) | SEQ ID NO: 14, | SEQ ID NO: 30, and | SEQ ID NO: 50; |
| xxiii.) | SEQ ID NO: 15, | SEQ ID NO: 31, and | SEQ ID NO: 51; |
| xxiv.) | SEQ ID NO: 16, | SEQ ID NO: 32, and | SEQ ID NO: 52; |
| xxv.) | SEQ ID NO: 17, | SEQ ID NO: 33, and | SEQ ID NO: 53; |
| xxvi.) | SEQ ID NO: 18, | SEQ ID NO: 34, and | SEQ ID NO: 54; and |
| xxvii.) | SEQ ID NO: 19, | SEQ ID NO: 35, and | SEQ ID NO: 55. |

2. The protein of claim 1, comprising one or more of nanobody amino acid sequences set forth in SEQ ID NOS: 56-83.

3. The protein of claim 2, comprising one or more of the huGRP78 binding nanobody amino acid sequences set forth in SEQ ID NO:67, SEQ ID NO:70, SEQ ID NO: 73, SEQ ID NO:75, and SEQ ID NO:82.

4. The protein of claim 2, comprising one or more of the huGRP78 binding nanobody amino acid sequences set forth in SEQ ID NO:67 and SEQ ID NO:73.

5. The protein of claim 1, consisting essentially of a single VHH domain.

6. The protein of claim 1, wherein the protein is a nanobody Fc fusion protein.

7. A pharmaceutical composition comprising the protein of claim 1 and at least pharmaceutically acceptable excipient.

8. A radiopharmaceutical composition comprising the protein of claim 1 linked to a radionuclide.

9. The radiopharmaceutical composition of claim 8, further comprising at least one pharmaceutically acceptable excipient.

10. The radiopharmaceutical composition of claim 9, wherein the radionuclide is an alpha particle emitter.

11. The radiopharmaceutical composition of claim 9, wherein the radionuclide is a beta particle emitter.

12. The radiopharmaceutical composition of claim 9, wherein the radionuclide comprises ¹³¹I.

13. The radiopharmaceutical of claim 9, wherein the radionuclide comprises ²²⁵Ac, ¹⁷⁷Lu or ⁹⁰Y.

14. A composition comprising the protein of claim 1, chemically conjugated to a chelator.

15. The composition of claim 14, wherein the chelator comprises DOTA or a DOTA derivative.

16. The composition of claim 14, further comprising a radionuclide chelated by the chelator.

17. The composition of claim 15, further comprising a radionuclide chelated by the chelator.
